(19) United States
(12) Patent Application Publication
(10) Pub. No.: US 2025/0040880 A1
(43) Pub. Date: Feb. 6, 2025

(54) IDENTIFYING SUITABLE CANDIDATES FOR DENERVATION THERAPY

(71) Applicant: Medtronic Ireland Manufacturing Unlimited Company, Dublin (IE)

(72) Inventors: Darion Peterson, Longmont, CO (US); Douglas A. Hettrick, Andover, MN (US)

(21) Appl. No.: 18/717,328

(22) PCT Filed: Nov. 21, 2022
(86) PCT No.: PCT/EP2022/082657

§ 371 Date: Jun. 6, 2024

Related U.S. Application Data
(60) Provisional application No. 63/265,173, filed on Dec. 9, 2021.

(51) Int. Cl. A61B 5/00; A61B 5/02; A61B 18/14; A61K 31/353

(52) U.S. Cl. CPC A61B 5/4836; A61B 5/02007; A61B 18/1492; A61K 31/353

(57) ABSTRACT

A computing device is configured to identify a suitable patient candidate for denervation therapy. The therapeutic assembly includes an energy delivery element. The computing device is configured to obtain, for each location of a plurality of locations of a vessel of a patient, a corresponding indication of stiffness associated with the location of the vessel. The computing device is further configured to determine whether the corresponding indications of stiffness satisfy a homogeneity condition. Responsive to the computing device determining that the corresponding indications of stiffness satisfy the homogeneity condition, the computing device is further configured to output an indication that the patient is a candidate for a denervation therapy.

### TECHNICAL FIELD

This disclosure generally relates to techniques for identifying patients who are likely to be responsive to denervation therapy.

### BACKGROUND

Percutaneous renal denervation is a minimally invasive procedure that can be used for treating hypertension. During a renal denervation procedure, a clinician delivers stimuli or energy, such as radiofrequency, ultrasound, cooling or other energy, to a treatment site to reduce activity of nerves surrounding a blood vessel. The stimuli or energy delivered to the treatment site may provide various therapeutic effects through alteration of sympathetic nerve activity.

### SUMMARY

The present disclosure describes devices, systems, and methods for identifying a suitable candidate for denervation therapy. In some examples, a computing device is configured to: obtain, for each location of a plurality of locations of a vessel of a patient, a corresponding indication of stiffness associated with the location of the vessel; determine whether the corresponding indications of local vessel stiffness satisfy a homogeneity condition; and responsive to the computing device determining that the corresponding indications of stiffness satisfy the homogeneity condition, output an indication that the patient is a candidate for a denervation therapy.

In some examples, a system comprises: a generator configured to deliver denervation therapy to a patient; a denervation device coupled to the generator; a data collection device configured to collect data indicative of vessel stiffness; and a computing device communicatively coupled to the data collection device, wherein the computing device is configured to: obtain, for a location of a vessel of the patient and at a first time, a first corresponding indication of stiffness; obtain, for the location and at a second time, a second corresponding indication of stiffness, wherein the second time is subsequent to the first time; determine a difference between the first corresponding indication of stiffness and the second corresponding indication of stiffness; and output an indication to end the delivery of denervation therapy in response to the difference satisfying a delivery condition.

In some examples, a method comprising: obtaining, by a computing device, for each location of a plurality of locations of a vessel of a patient, a corresponding indication of a stiffness of the associated with the location of the vessel; determining, by the computing device, whether the corresponding indications of stiffness satisfy a homogeneity condition; and in response to determining that the corresponding indications of stiffness satisfy the homogeneity condition, outputting, by the computing device, an indication that the patient is a candidate for a denervation therapy.

In some examples, a method comprising: determining, for a location of a vessel of a patient and at a first time, a first corresponding indication of stiffness; delivering denervation therapy to the patient; determining, for the location of the vessel and at a second time, a second corresponding indication of stiffness, wherein the second time is subsequent to the first time; determining a difference between the first corresponding indication of stiffness and the second corresponding indication of stiffness; and ending the delivery of renal denervation therapy in response to the difference satisfying a threshold value.

Also disclosed herein is a computing device that is configured to identify a suitable patient candidate for denervation therapy, wherein the therapeutic assembly includes an energy delivery element, wherein the computing device is configured to obtain, for each location of a plurality of locations of a vessel of a patient, a corresponding indication of stiffness associated with the location of the vessel, wherein the computing device is further configured to determine whether the corresponding indications of stiffness satisfy a homogeneity condition, and wherein, responsive to the computing device determining that the corresponding indications of stiffness satisfy the homogeneity condition, the computing device is further configured to output an indication that the patient is a candidate for a denervation therapy.

Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 is a conceptual diagram illustrating an example system for identifying suitable patient candidates for denervation therapy as well as delivering denervation therapy, in accordance with some examples of the current disclosure.

FIG. 2 is a block diagram illustrating an example configuration of a data collection device, in accordance with some examples of the current disclosure.

FIGS. 3A-3C are conceptual diagrams of a sequence of pulse wave imaging images, in accordance with some examples of the current disclosure.

FIG. 4 is a conceptual diagram of an example of a denervation device in an expanded deployed configuration within a vessel of a patient.

FIG. 5 is a block diagram illustrating an example configuration of a generator.

FIG. 6 is a flow diagram illustrating an example technique for operating a system to identify suitable patient candidates for denervation therapy.

FIG. 7 is a flow diagram illustrating an example technique for operating a system to identify suitable patient candidates for denervation therapy.

FIG. 8 is a flow diagram illustrating an example technique for operating a system to deliver denervation therapy.

### DETAILED DESCRIPTION

Denervation therapy, such as renal denervation therapy, may be used to render a nerve inert, inactive, or otherwise completely or partially reduced in function, such as by ablation or lesioning of the nerve. Following denervation, there may be a reduction or even prevention of neural signal transmission along the target nerve. Denervating an overactive nerve may provide a therapeutic benefit to a patient. For example, renal denervation may mitigate symptoms associated with renal sympathetic nerve overactivity. Denervation therapy may include delivering electrical and/or thermal energy to a target nerve, and/or delivering a chemical agent to a target nerve. In the case of renal denervation therapy, the denervation energy or chemical agents can be delivered, for example, via a therapy delivery device (e.g., a catheter) disposed in a blood vessel (e.g., the renal artery) proximate the renal nerve.

The renal sympathetic nervous system has been identified as a major contributor to the complex pathophysiology of hypertension, or elevated systemic blood pressure. Therefore, renal denervation may reduce renal sympathetic nerve overactivity and cause a reduction in systemic blood pressure as a treatment for hypertension. In some patients, renal denervation may reduce systolic blood pressure in a range of approximately 5 millimeters of mercury (mmHg) to 30 mmHg. However, renal denervation may not reduce systemic blood pressure for other patients.

Efforts to identify candidates who might respond better to the renal denervation therapy have focused in two primary areas. First, identification of patients with potentially higher baseline sympathetic activity as indicated by variables such as increased heart rate, increased plasma renin levels, increased muscle sympathetic nerve activity, increased renal norepinephrine spillover, or the like has been suggested. Second, identification of higher baseline aortic or arterial stiffness due to calcification or other vascular disease that would prevent peripheral arterial vasodilation has also been suggested. Various methods to quantify aortic/arterial stiffness in patients otherwise eligible for renal denervation have been attempted, including imaging based methods of estimating aortic distensibility, invasive determination of pulse wave velocity, noninvasive determination of pulse wave velocity using either two point (Ad/At) techniques or morphometry model based single beat estimates, estimates of arterial wave reflection such as an augmentation index, or high arterial pulse pressure (i.e. “isolated systolic hypertension”) identified as high systolic blood pressure with low or normal diastolic blood pressure. However, none of these techniques to date has become well established as an accurate and reliable predictor of renal denervation responders.

In accordance with techniques described herein, candidates who are likely to respond better to renal therapy may be identified by distinguishing between irreversible structural increases in arterial stiffness, such as calcification and other vascular disease, versus dynamically increased aortic or arterial stiffness primarily due to sympathetically mediated increases in arterial smooth muscle thickness or tone. In some examples, pulse wave imaging may be used to measure pulse wave velocity, and thus an indication of arterial stiffness, in a localized manner. In contrast to some other pulse transit time techniques, pulse wave imaging does not integrate the pulse wave velocity measurement over a long distance and does not determine pulse wave velocity as a single number. Instead, in pulse wave imaging, pulse wave velocities are determined for each of a plurality of relatively specific, localized areas of the vessel. This allows identification of heterogeneous structural variabilities within the vessel, such as those that might be seen with extravascular calcification or plaque, aneurysm, vessel dissection, or the like. Conversely, this also allows identification of dynamic local causes for increased pulse wave velocity, such as increased smooth muscle tone or thickness, which will appear more homogeneous. A patient with more homogeneous vessel wall stiffness may be a better candidate for renal denervation, as they may be more likely to respond to renal denervation.

In this way, by using pulse wave imaging or another technique to obtain, for each location of a plurality of locations of a vessel of a patient, a corresponding indication of stiffness associated with the location of the vessel, and determining whether the corresponding indications of stiffness satisfy a homogeneity condition, the techniques described herein may allow identification of candidates for renal denervation.

Alternatively, or additionally, pulse wave imaging or another technique for determining indications of stiffness associated with the locations along or within a vessel may be used to determine whether a denervation therapy has been successful. For instance, a first pulse wave imaging procedure may be performed prior to denervation therapy. After the denervation therapy has been performed, a second pulse wave imaging procedure may be performed. Results from the second pulse wave imaging procedure may be compared to results from the first pulse wave imaging procedure and compared to a threshold. The difference satisfying the threshold may indicate that the denervation therapy is successful.

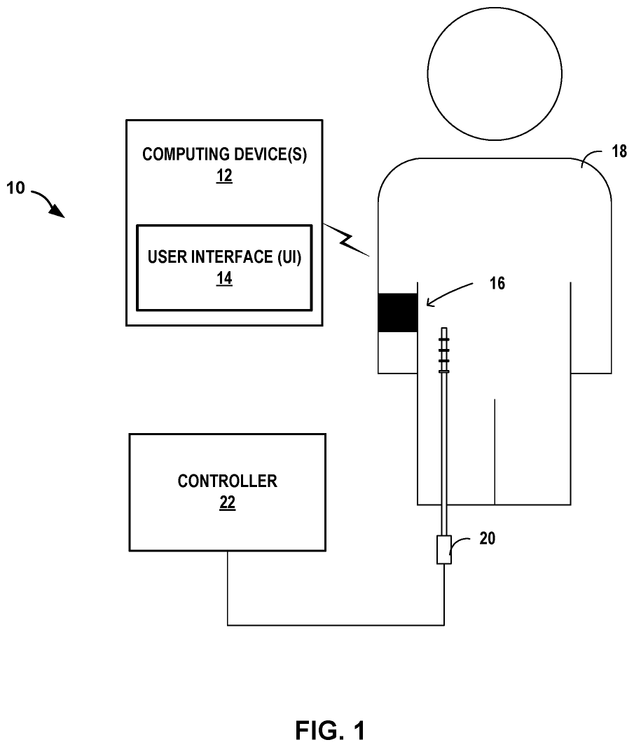

FIG. 1 is a conceptual diagram illustrating an example system 10 for identifying patient candidates for denervation therapy as well as, in some examples, delivering denervation therapy. As shown in FIG. 1, system 10 includes a computing device 12. Computing device 12 may be a computing device used in a home, ambulatory, clinic, or hospital setting. Computing device 14 may include, for example, a clinician programmer, a desktop computer, a laptop computer, a workstation, a server, a mainframe, a cloud computing system, combinations thereof, or the like. Computing device 12 may be configured to receive, via a user interface device 14 (“UI 14”), input from a user, such as a clinician, output information to a user, or both. In some examples, UI 14 may include a display (e.g., a liquid crystal display (LCD) or light emitting diode (LED) display), such as a touch-sensitive display; one or more buttons; one or more keys (e.g., a keyboard); a mouse; one or more dials; one or more switches; a speaker; one or more lights; combinations thereof; or the like.

Computing device 12 may be communicatively coupled to a data collection device 16. Data collection device 16 may be configured to collect data indicative of vessel stiffness associated with one or more locations of a vessel of patient 18. In some examples, data collection device 16 may include an ultrasound system. An ultrasound system may include one or more ultrasound transducer and an ultrasound controller. The ultrasound transducer may be configured for external use or configured for intravascular use. In some such examples, data collection device 16 may advantageously serve as a tool for enabling noninvasive or minimally invasive characterization of vessel wall properties, such as stiffness. Data collection device 16 may collect data associated with a plurality of locations of the vessel. Data collection device 16 may be configured to communicate the data to computing device 12.

Data collection device 16 may be configured to collect data representative of a physiological parameter associated a portion (e.g., a vessel) of a patient 18. For instance, data collection device 16 may include an array of one or more ultrasound transducers configured to deliver, into tissue of patient 18, ultrasound pulses, which reflect and diffract off the tissue. The array of ultrasound transducers may receive the reflected and diffracted ultrasound pulses, enabling visualization of internal body structures (e.g., tendons, muscles, joints, vessels, internal organs, and the like).

In some implementations, system 10 may be configured to perform denervation within the vessel of patient 18. System 10 may perform the denervation endovascularly, intravascularly, or externally from patient 18. System 10 may include a denervation device 20 and a controller 22. Denervation device 20 may include any device that delivers energy or stimulus to a target nerve within a wall of a blood vessel, such as the renal nerve of the renal artery. In some implementations, denervation device 20 may be a device positioned external to patient 18, such as a transducer that emits ultrasound energy. In some examples, data collection device 16 may be configured to also function as denervation device 20.

Alternatively, denervation device 20 may be configured to be intravascularly positioned within a vessel or other anatomical lumen to deliver the energy or stimulus. The energy or stimulus may include, for example, at least one of a radio frequency (RF) stimulus, a thermal stimulus, a cryogenic stimulus, a microwave stimulus, an ultrasonic stimulus, or other form of energy or stimulus.

As described in greater detail below, in some examples, denervation device 20 may include a catheter and/or one or more energy delivery elements, such as an electrode, and/or one or more sensors. The catheter may be intravascularly delivered into patient 18, e.g., into a vessel of patient 18, in a low-profile configuration, such as the substantially straight configuration shown in FIG. 1. Upon delivery to a target location within and along the vessel, the catheter may be deployed into an expanded deployed configuration, such as a generally helical or spiral configuration or other suitable configuration, that causes the one or more energy delivery elements, such as one or more electrodes, to contact portions of the vessel wall. In the expanded deployed configuration, denervation device 20 may deliver energy at a treatment site and provide therapeutically-effective electrical and/or thermally induced denervation to a nerve within the vessel.

In accordance with techniques of this disclosure, computing device 12 may be configured to obtain, for each location of a plurality of locations of a vessel of patient 18, a corresponding indication of stiffness associated with the location of the vessel. The corresponding indications of stiffness may be based on the data received from data collection device 16. In some examples, computing device 12 may obtain the corresponding indications of stiffness by computing corresponding pulse wave velocity (PWV) values associated with each of a plurality of locations of a vessel (e.g., artery, vein, or the like). As used herein, PWV refers to a value representing the velocity of the pressure and flow waves that propagate through blood vessels of a patient because of ventricular ejection.

In general, a specific location of a vessel may be associated with at least one of a specific longitudinal location or a specific circumferential location of the vessel. In an example, a specific location of a vessel is only associated with a specific longitudinal location of the vessel. In another example, a specific location of a vessel is only associated with a specific circumferential location of the vessel. In yet another example, a specific location of a vessel is associated with both a specific longitudinal location and a specific circumferential location of the vessel.

In some implementations, the PWV values may be derived from pulse wave imaging (PWI), an ultrasound imaging modality that tracks pulse wave propagation through a vessel. In such implementations, data collection device 16 may image a vessel segment at a high sampling frequency (e.g., tens of times per second) (e.g., by using a 3- or 5-plane wave compounding acquisition sequence). In some examples, data collection device 16 communicates the image data to computing device 12, which processes the image data to determine, for each location of the plurality of locations, a corresponding localized pulse wave velocity measured using pulse wave imaging. In other examples, data collection device 16 determines, for each location of the plurality of locations, a corresponding localized pulse wave velocity measured using pulse wave imaging and communicates the corresponding localized pulse wave velocities to computing device 12.

Computing device 12 or data collection device 16 may apply any suitable image analysis technique to determine the propagation of the pulse wave and localized pulse wave velocities. For instance, computing device 12 or data collection device 16 may apply a speckle tracking technique on the received ultrasound image data to determine the propagation of the pulse wave at each of a plurality of locations of the vessel, and, thus, corresponding localized pulse wave velocities associated with the plurality of locations of the vessel. Regardless, computing device 12 obtains, for each location of the plurality of locations, a corresponding localized pulse wave velocity measured using pulse wave imaging.

In another example, data collection device 16 may be configured to obtain the corresponding indications of vessel wall stiffness by using shear-wave elastography (SWE). As used herein, SWE refers to an ultrasound-based technique for quantifying the mechanical properties of tissue by measuring waves that travel laterally and perpendicularly to emitted ultrasound pulses. SWE may involve using an acoustic radiation force pulse sequence to generate shear waves, which propagate perpendicular to the ultrasound pulses, causing transient displacements. In this way, computing device 12 may be configured to receive, for each location of the plurality of locations, a corresponding shear wave speed measured using ultrasound shear wave elastography. The distribution of shear-wave velocities in tissue may be directly related to the shear modulus of the tissue, an absolute measure of the tissue's elastic properties. For instance, a faster wave velocity may be related to a higher tissue modulus. Hence, SWE may enable direct calculation of vessel stiffness. When using SWE, computing device 12 may obtain, for each location of the plurality of locations, a direct indication of vessel stiffness.

In any case, computing device 12 may obtain the corresponding indications of vessel wall stiffness for the plurality of locations of the vessel. To determine whether patient 18 is a suitable candidate for denervation therapy, computing device 12 may be further configured to determine whether the corresponding indications of vessel wall stiffness satisfy a homogeneity condition. For instance, as described above, a patient with a more homogeneous distribution of vessel wall stiffness among the locations may be a better candidate for renal denervation (i.e., more likely to respond to renal denervation therapy), while a patient with a more heterogeneous distribution of vessel wall stiffness among the locations may be a poorer candidate for renal denervation (i.e., less likely to respond to renal denervation therapy).

Satisfaction of the homogeneity condition may indicate more similar vessel stiffness values (e.g., substantially similar stiffness values) associated with the plurality of locations of the vessel. In some implementations, the corresponding indications of stiffness may satisfy the homogeneity condition when a range of the vessel stiffness values (i.e., the difference between the largest vessel stiffness value and the smallest vessel stiffness value) indicated by the corresponding indications of stiffness is less than or equal to a threshold value associated with the homogeneity condition. In other implementations, the corresponding indications of stiffness may satisfy the homogeneity condition when a standard deviation or variance among the indications of stiffness is less than a threshold value.

As an example, a first indication of stiffness associated with a first location of a vessel may indicate a pulse wave velocity of about 4.5 m/s, a second indication of stiffness associated with a second location of a vessel may indicate a pulse wave velocity of about 4.8 m/s, and a third indication of stiffness associated with a third location of a vessel may indicate a pulse wave velocity of about 4.6 m/s. The range of the corresponding indications of stiffness may therefore be 0.3 m/s; accordingly, if the threshold value is 0.5 m/s or greater, then the range of the corresponding indications of stiffness may satisfy the homogeneity condition. However, if the threshold value is 0.2 m/s, then the range of the corresponding indications of stiffness may not satisfy the homogeneity condition. In any case, responsive to computing device 12 determining that the corresponding indications of stiffness satisfy the homogeneity condition, computing device 12 may output an indication that patient 18 is a candidate for a denervation therapy. Conversely, responsive to computing device 12 determining that the corresponding indications of stiffness does not satisfy the homogeneity condition, computing device 12 may output an indication that patient 18 is not a candidate for a denervation therapy.

In some implementations, computing device 12 may determine satisfaction of the homogeneity condition based on other conditions and/or determinations. For instance, instead of directly determining whether the corresponding indications of stiffness satisfy a homogeneity condition, computing device 12 may determine whether the corresponding indications of stiffness do not satisfy a heterogeneity condition. Satisfaction of the heterogeneity condition may indicate substantially different vessel stiffness values associated with the plurality of locations of the vessel. As such, nonsatisfaction of the heterogeneity condition may indicate substantially similar vessel stiffness values associated with the plurality of locations of the vessel, which may imply satisfaction of the homogeneity condition.

In some examples, computing device 12 may be configured to determine whether patient 18 is a candidate for denervation therapy based on one or more conditions in addition (or as an alternative) to the homogeneity condition or equivalents thereof. For example, computing device 12 may be configured to determine whether patient 18 is a candidate for denervation therapy based on a difference condition, which relates to a comparison between the plurality of indications of vessel stiffness and a second plurality of indications of vessel stiffness after a perturbation of blood flow or the sympathetic nervous system. In particular, computing device 12 may determine that patient 18 is a viable candidate when a difference between a first corresponding plurality of indications of stiffness for a plurality of locations of a vessel and a second corresponding plurality of indications of stiffness for the plurality of locations of the vessel satisfies the difference condition.

As an example, computing device 12 may obtain, via data collection device 16 and as described above, a first corresponding plurality of indications of stiffness for the plurality of locations of the vessel. Additionally, computing device 12 may obtain a second corresponding plurality of indications of stiffness for the plurality of locations of the vessel in response to a perturbation of blood flow through the vessel. Perturbation of blood flow may be induced or otherwise caused by a bolus injection of a catecholamine, an acute occlusion of an inferior vena cava, a cold pressor stimulation, a Valsalva maneuver, a Müller's maneuver, a hand grip exercise, and/or the like.

In cases where vessel stiffness is reversible (e.g., due to sympathetic nerve overactivity), perturbation of blood flow may result in a change in vessel stiffness, usually by decreasing the vessel stiffness. As such, if the second corresponding plurality of indications of stiffness, which computing device 12 obtains in response to perturbation, is sufficiently different from first corresponding plurality of indications of stiffness, that difference may indicate that patient 18 has reversible vessel stiffness, which may in turn indicate that patient 18 is a suitable candidate for denervation therapy. Accordingly, computing device 12 may determine whether patient 18 is a suitable candidate for denervation therapy based on whether the difference satisfies the difference condition. In some examples, computing device 12 may determine that the difference satisfies the difference condition if the difference is equal to or greater than a threshold value associated with the difference condition.

In some examples, computing device 12 may determine a respective result of the difference condition for each location of the plurality of locations. Computing device 12 then may determine whether a certain amount (e.g., fraction or percentage) of the plurality of locations satisfies the difference condition. In other examples, computing device 12 may determine a result of the difference condition for a subset of locations of the plurality of locations. Computing device 12 then may determine whether a certain amount (e.g., fraction or percentage) of the plurality of locations satisfies the difference condition. Alternatively, computing device 12 may determine a result of the difference condition for one location of the plurality of locations. In some implementations, the locations associated with the second plurality of indications of stiffness may not exactly match the locations associated with the first plurality of indications of stiffness, as the measurements are done at a different time such that the vessel may be in a different position.

In some implementations, computing device 12 may determine whether patient 18 is a suitable candidate based on whether the second plurality of corresponding indications of stiffness satisfies a maximum condition. In some examples, computing device 12 may determine that the second plurality of corresponding indications of stiffness satisfies the maximum condition if the second plurality of corresponding indications of stiffness (or a statistical derivation thereof, such as a mean, median, mode, etc.) is equal to or greater than a threshold value associated with the maximum condition.

It should be understood that computing device 12 may use one or more conditions to determine whether patient 18 is a suitable candidate for denervation therapy. For example, computing device 12 may output an indication that patient 18 is a candidate for denervation therapy in response to satisfaction of only the homogeneity condition. In another example, computing device 12 may output an indication that patient 18 is a candidate for denervation therapy in response to satisfaction of the homogeneity condition and the difference condition. In yet another example, computing device 12 may output an indication that patient 18 is a candidate for denervation therapy in response to satisfaction of the homogeneity condition, the difference condition, and the maximum condition. Other configurations are contemplated by this disclosure.

Alternatively, or additionally, computing device 12 may be configured to determine whether a denervation procedure has been successful using one or more indications of vessel stiffness. For instance, data collection device 16 may be used to perform a first pulse wave imaging procedure (or other measure of vessel stiffness) prior to a denervation therapy. This may generate a first plurality of indications of vessel stiffness. Controller 22 and denervation device 20 then may be used to perform a denervation therapy, e.g., using RF energy, chemical ablation, cryoablation, ultrasound, microwave energy, or the like. After the denervation therapy has been performed, data collection device 16 may be used to perform a second pulse wave imaging procedure (or other measure of vessel stiffness). This may generate a second plurality of indications of vessel stiffness. Computing device 12 may be configured to compare results from the second pulse wave imaging procedure to results from the first pulse wave imaging procedure. For instance, computing device 12 may be configured to compare a statistical value (e.g., mean, median, or the like) derived from the second plurality of indications of vessel stiffness and a statistical value (e.g., mean, median, or the like) derived from the first plurality of indications of vessel stiffness. For example, computing device 12 may determine a difference between the statistical value (e.g., mean, median, or the like) derived from the second plurality of indications of vessel stiffness and the statistical value (e.g., mean, median, or the like) derived from the first plurality of indications of vessel stiffness. Computing device 12 may be configured to compare the difference to a threshold value. Computing device 12 may be configured indicate that the denervation therapy is successful in response to the difference satisfying the threshold. Conversely, computing device 12 may be configured indicate that the denervation therapy is unsuccessful in response to the difference not satisfying the threshold. In this way, indications of vessel stiffness may be used to evaluate efficacy of a denervation therapy, such as a renal denervation therapy.

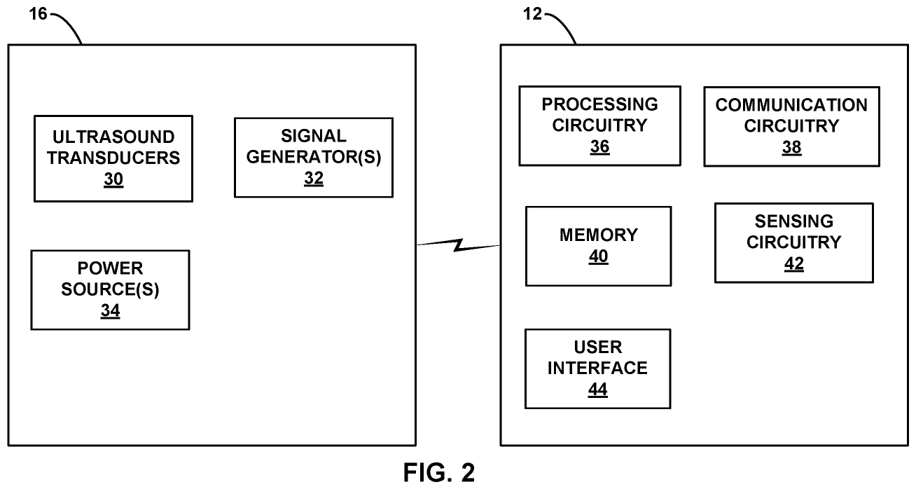

FIG. 2 is a block diagram illustrating an example configuration of data collection device 16. As shown in FIG. 2, data collection device 16 includes one or more ultrasound transducers 30 (e.g., arranged in an array), one or more signal generators 32 for driving ultrasound transducers 30 to deliver ultrasound energy, and one or more power sources 34 that provide power to the one or more signal generators 32 for driving ultrasound transducers 30, as well as providing power to other components of data collection device 16.

As also shown in FIG. 2, computing device 12 includes processing circuitry 36, communication circuitry 38, memory 40, sensing circuitry 42, and UI 14. Memory 40 may include any volatile or non-volatile media, such as a random access memory (RAM), read only memory (ROM), non-volatile RAM (NVRAM), electrically erasable programmable ROM (EEPROM), flash memory, or the like. Memory 40 may store computer-readable instructions that, when executed by processing circuitry 36, cause computing device 12 to perform various functions described herein. Processing circuitry 36 may include any combination of one or more processors including one or more microprocessors, digital signal processors (DSPs), application specific integrated circuits (ASICs), field programmable gate arrays (FPGAs), or other equivalent integrated or discrete logic circuitry. Accordingly, processing circuitry 36 may include any suitable structure, whether in hardware, software, firmware, or any combination thereof, to perform the functions ascribed herein to processing circuitry 36 and data collection device 16.

Processing circuitry 36 may be configured to control signal generators 32 to output a signal to ultrasound transducers 30 to cause ultrasound transducers 30 to deliver ultrasound energy for an imaging purpose and, in some examples, a therapeutic purpose. In some implementations, processing circuitry 36 may control signal generators 32 to generate a signal using power from power sources 34 that drives ultrasound transducers 30 to deliver ultrasound energy. Signal generators 32 may include one or more oscillators configured to generate signals of a desired frequency for the ultrasound energy, amplification or other circuitry to control the amplitude of the driving signals, as well as switching circuitry to selectively direct the signal to one or more of ultrasound transducers 30 and/or selectively control the on/off state of individual ones or sets of transducers 30. Some or all of the circuitry associated with signal generators 32 may be respectively associated with certain sets of ultrasound transducers 30, or circuitry associated with signal generators 32 may be shared by all or a subset of ultrasound transducers 30.

Processing circuitry 36 may control signal generators 32 to output a signal to ultrasound transducers 30 to deliver ultrasound energy to a particular depth, region, or point of tissue, with a particular amplitude, by selecting which of ultrasound transducers 30 is on or driven, and controlling one or more of the amplitude or phase of the driving signal provided to the driven ultrasound transducers 30 by signal generators 32. Different active ultrasound transducers 30 or sets of ultrasound transducers may be driven with different signals, e.g., different amplitudes and/or phases, to target a desired, depth, region, or point of tissue.

Sensing circuitry 42 may be configured to selectively (e.g., as controlled by processing circuitry 36) receive and condition electrical signals produced by ultrasound transducers 30 in response to reflected ultrasound, for processing by processing circuitry 36. Sensing circuitry 42 may include one or more switches to control which one or more of transducers 30 are active to sense reflected ultrasound.

Power sources 34 may deliver operating power to various components of data collection device 16. Power sources 34 may include a rechargeable or a non-rechargeable battery and a power generation circuit to produce the operating power. Recharging may be accomplished through proximal inductive interaction between a charging device and an inductive charging coil of data collection device 16, or a wired connection between the charging device and data collection device 16.

Communication circuitry 36 is configured to support wired and/or wireless communication between computing device 12 and one or more other devices, such as data collection device 16. A user may control the delivery of ultrasound energy by data collection device 16, as well as the collection of imaging ultrasound and/or sensing by data collection device 16, via communication with processing circuitry 36 of computing device 12 through communication circuitry 36. In some examples, programs that control the delivery of ultrasound energy, collection of imaging ultrasound, and/or sensing may be stored in memory 40 and executed by processing circuitry 36. Ultrasound images and other such information may be stored in memory 40.

UI 14 may include an input device and an output device. The input device may be a user interface element, such as a button, a dial, a microphone, a keyboard, a touch screen, or the like. The output device may be a display, a speaker, an audio and/or visual indicator, or the like. The output device may display an alert or notification or other information to the clinician, such as whether patient 18 is a candidate for denervation therapy. In some examples, the output device may be an audio output device that outputs an audio indicator that indicates the notification or information to the clinician.

Computing device 12 may be configured to receive data (e.g., via communication circuitry 36) representative of an image of a vessel from data collection device 16. As described above, the image may include a pulse wave imaging image, a shear-wave elastography (SWE) image, or the like. In some examples, the image may be obtained using a contrast enhanced ultrasound. Contrast enhanced ultrasound may facilitate imaging of a vessel wall, e.g., by enhancing contrast between the vessel lumen and adjacent tissue (the vessel wall). This may facilitate analysis of the image data by computing device 12 to identify pulse wave velocities or other indications of vessel stiffness.

In some examples, in addition to determining or allowing determination of a plurality of indications of stiffness associated with locations of a vessel, the image data obtained by computing device 12 (using data acquisition device 16), the image data may allow identification of whether at least one of plaque, extravascular calcification, aneurysm, or vessel dissection is present within the vessel. For instance, computing device 12 may output the image via the output device (e.g., a display) of UI 14. Based on the image of the vessel, a clinician or other user of system 10 may discriminate between reversible vessel stiffness (in which the at least one of plaque, extravascular calcification, aneurysm, or vessel dissection is not present within the vessel) and irreversible vessel stiffness (in which at least one of plaque, extravascular calcification, aneurysm, or vessel dissection is present within the vessel). That is, in some cases, the clinician (or another user) may discriminate between reversible vessel stiffness and irreversible vessel stiffness based on identifying, in the image of the vessel, at least one of plaque, extravascular calcification, aneurysm, or vessel dissection within the vessel. Alternatively, computing device 12 may perform image analysis to identify at least one of plaque, extravascular calcification, aneurysm, or vessel dissection within the vessel.

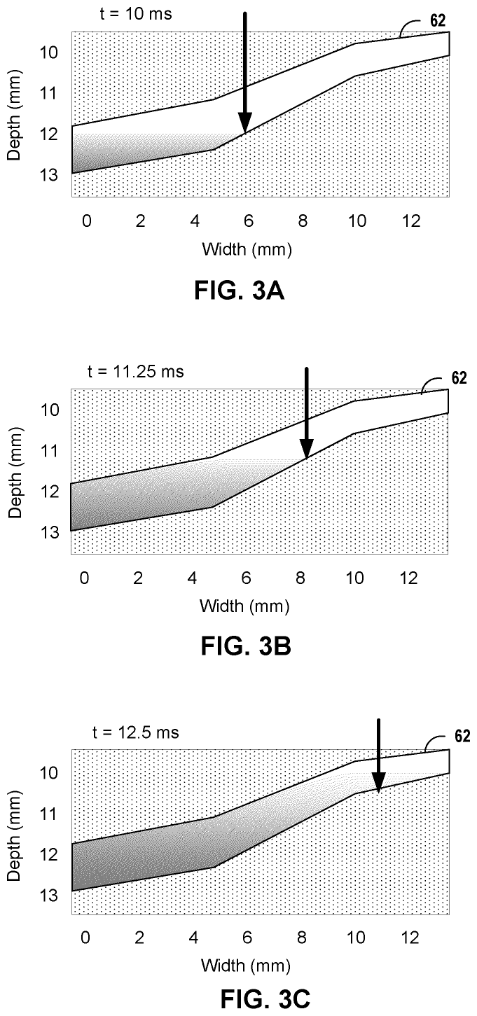

As described above, computing device 12 may be configured to analyze a plurality of images to determine a plurality of pulse wave velocities, which may serve as indications of vessel wall stiffness. FIGS. 3A-3C are conceptual diagrams illustrating a sequence of pulse wave imaging images. In particular, FIG. 3A is an illustration of an image of a pulse wave (moving from left to right and represented by a gray gradient) propagating through a vessel 62 at a time, t₁; FIG. 3B is an illustration of an image of the pulse wave propagating through vessel 62 at a time t₂; and FIG. 3C is an illustration of an image of the pulse wave propagating through vessel 62 at a time t₃. The arrows in FIGS. 3A-3C indicate the propagation of the pulse wave through vessel 62. As noted above, pulse wave imaging techniques may be used to determine the pulse wave velocity in a specific localized area of a vessel, where the pulse wave velocity might differ among the locations. In general, computing device 12 may calculate the pulse wave velocity by dividing the displacement of the pulse wave through vessel 62 by the change in time.

In the example of FIG. 3A, the pulse wave image at the first location of vessel 62 may indicate a first pulse wave velocity. In the example of FIG. 3B, the pulse wave image at the second location of vessel 62 may indicate a second pulse wave velocity, which may be the same as or different from the first pulse wave velocity. In the example of FIG. 3C, the pulse wave image may indicate a third pulse wave velocity, which may be the same as or different from the first pulse wave velocity and/or the second pulse wave velocity. Computing device 12 may analyze the first, second, and third pulse wave velocities to determine if the first, second, and third pulse wave velocities satisfy the homogeneity criterion. In any case, responsive to computing device 12 determining that the corresponding indications of stiffness satisfy the homogeneity condition, computing device 12 may output an indication that patient 18 is a candidate for a denervation therapy; alternatively, responsive to computing device 12 determining that the corresponding indications of stiffness does not satisfy the homogeneity condition, computing device 12 may output an indication that patient 18 is a not candidate for a denervation therapy.

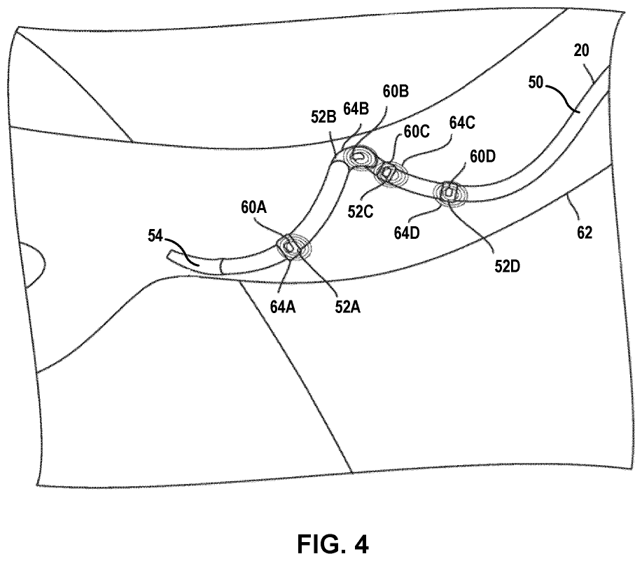

In some examples, in response to determining that patient 18 is a candidate for a denervation therapy, system 10 may be used to deliver renal denervation therapy. For instance, FIG. 4 is a conceptual diagram of an example of denervation device 20. Denervation device 20 includes an elongated body 50 (e.g., a catheter body). In the example shown in FIG. 4, elongated body 50 is in an expanded deployed configuration within vessel 62 of patient 18. In some examples, vessel 62 may be an artery, such as an aorta, a renal artery, a splanchnic artery, a hepatic artery, a mesenteric artery, or the like. Elongated body 50 extends a proximal end to a distal end 54. Elongated body 50 carries energy delivery elements 52A-52D (collectively, “energy delivery elements 52”). Elongated body 50 may be introduced into and advanced along vessel 62 to position energy delivery elements 52 within a target vessel and deployed so that energy delivery elements 52 contact a wall of vessel 62 in the expanded deployed configuration. In some implementations, denervation device 20 may further carry one or more sensors, such as sensors 60A-60D (collectively, “sensors 60”). For instance, sensors 60 may include a temperature sensor.

For example, a first energy delivery element 52A may contact the wall of vessel 62 at a first location 64A, a second energy delivery element 52B may contact the wall of vessel 62 at a second location 64B, a third energy delivery element 52C may contact the wall of vessel 62 at a third location 64C, and a fourth energy delivery element 52D may contact the wall of vessel 62 at a fourth location 64D. Denervation device 20 may deliver energy through energy delivery elements 52 at the treatment site and provide therapeutically-effective electrically-induced denervation and/or thermally-induced denervation.

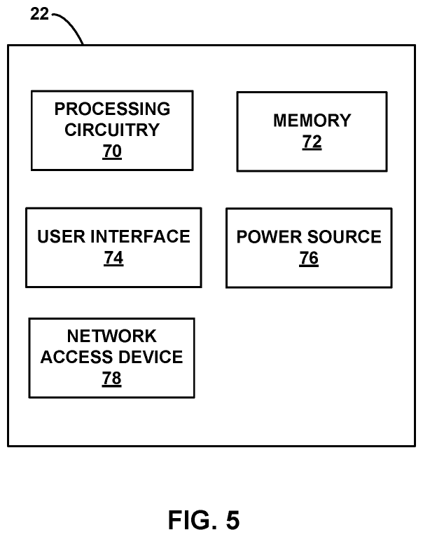

FIG. 5 is a block diagram illustrating an example configuration of controller 22, which may be configured to deliver electrical energy to energy delivery elements 52. Controller 22 may be a radio frequency generator or other electrical generator that generates and energy through energy delivery elements 52 to the wall of vessel 62 at the treatment location. Controller 22 may have a cable, an electrical lead and or wire that is electrically conductive and connects to one or more conductors that extends a length of denervation device 20 within a lumen and are electrically coupled with energy delivery elements 52. In some implementations, controller 22 may have separate leads and/or wires that electrically couple with a corresponding energy delivery element 52 of energy delivery elements 52 so that each of energy delivery elements 52 may operate independently of the others. For example, controller 22 may have multiple separate channels, such as four RF channels to deliver RF energy independently to energy delivery elements 52 and control and monitor each of energy delivery elements 52. Controller 22 may generate energy that ultimately is transmitted through the electrical lead to energy delivery elements 52.

As shown in FIG. 5, controller 22 includes processing circuitry 70, a memory 72, a user interface 74 (“UI 74”), a power source 76, and a network access device 78. Processing circuitry 70 may be electrically coupled to memory 72, UI 74 and/or power source 76. Processing circuitry 70 may control a state of each of energy delivery elements 52 and the amount of energy delivered to each of energy delivery elements 52 by power source 76 to manage the course and administration of treatment at the treatment site. Processing circuitry 70 may be coupled to memory 72 and execute instructions that are stored in memory 72.

Memory 72 may be coupled to processing circuitry 70 and store instructions that processing circuitry 70 executes. Memory 72 may include one or more of a RAM, ROM, or other volatile or non-volatile memory. Memory 72 may be a non-transitory memory or a data storage device, such as a hard disk drive, a solid-state disk drive, a hybrid disk drive, or other appropriate data storage, and may further store machine-readable instructions, which may be loaded and executed by processing circuitry 70.

Power source 76 may include a RF generator or other electrical source. Power source 76 may provide a selected form and magnitude of energy for delivery to the treatment site via denervation device 20. Controller 22 may include UI 74. Controller 22 may receive input, such as the selected form and the magnitude of energy to be delivered to each of energy delivery elements 52, via UI 74. UI 74 may receive other user input including the blood pressure of the human patient and/or the position of the human patient, such as when the human patient is in the supine position or the standing position.

UI 74 may include an input output device that receives user input from a user interface element, a button, a dial, a microphone, a keyboard, or a touch screen. UI 74 may provide an output to an output device, such as a display, a speaker, an audio and/or visual indicator, or a refreshable braille display. The output device may display an alert or notification or other information to the clinician and or to confirm status and or commands from the clinician. The output device may be an audio output device that outputs an audio indicator that indicates the notification or information to be provided to the clinician.

Network access device 78 may include a communication port or channel, such as one or more of a Dedicated Short-Range Communication (DSRC) unit, a Wi-Fi unit, a Bluetooth® unit, a radio frequency identification (RFID) tag or reader, or a cellular network unit for accessing a cellular network (such as 3G, 4G or 5G). In some implementations, network access device 78 may transmit data to and receive data from an external database or remote server.

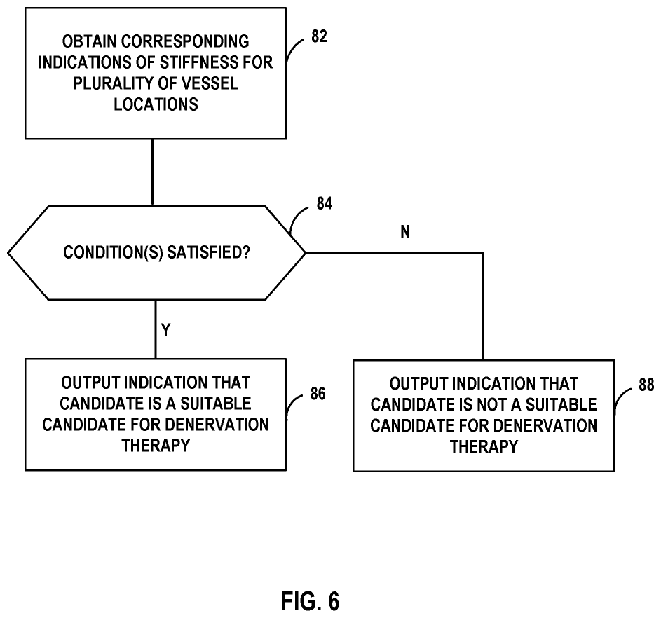

FIG. 6 is a flow diagram illustrating an example technique for operating system 10. As indicated by FIG. 6, computing device 12 may obtain corresponding indications of stiffness for a plurality of locations within vessel 62 (82). Computing device 12 may obtain the corresponding indications of stiffness from data collection device 16. In some examples, data collection device 16 may be configured to collect data representative of an image of a portion of vessel 62 of patient 18. For instance, data collection device 16 may include an array of one or more ultrasound transducers configured to deliver, into tissue of patient 18, ultrasound pulses. As described above, in some implementations, data collection device 16 may be configured to generate the indications of stiffness and communicate the indications of stiffness to computing device 12. In other implementations, data collection device 16 may be configured to collect image data, and computing device 12 may be configured to determine the indications of stiffness based on the image received from data collection device 16.

Computing device 12 may determine whether the corresponding indications of stiffness satisfy one or more conditions (84). The conditions may include a homogeneity condition, a difference condition, a maximum condition, or the like, or combinations thereof. If computing device 12 determines that the one or more conditions are satisfied (Y of 84), computing device 12 may output an indication that patient 18 is a suitable candidate for denervation therapy (86). However, if the one or more conditions are not satisfied (N of 84), computing device 12 may output an indication that patient 18 is not a suitable candidate for denervation therapy (88).

As an example, the one or more conditions may include a homogeneity condition and a difference condition. If the range of the vessel stiffness values indicated by the corresponding indications of stiffness is less than or equal to a threshold value for the homogeneity condition, then computing device 12 may determine that the homogeneity condition is satisfied. In addition, if the difference between a first corresponding plurality of indications of stiffness for a plurality of locations of a vessel and a second corresponding plurality of indications of stiffness for the plurality of locations of the vessel is greater than or equal to the difference condition, then computing device 12 may determine that the difference condition is satisfied.

In some examples, if both conditions are satisfied, computing device 12 may output an indication that patient 18 is a suitable candidate for denervation therapy (86). In some examples, if one or more of these conditions are not satisfied, computing device 12 may output an indication that patient 18 is not a suitable candidate for denervation therapy (88).

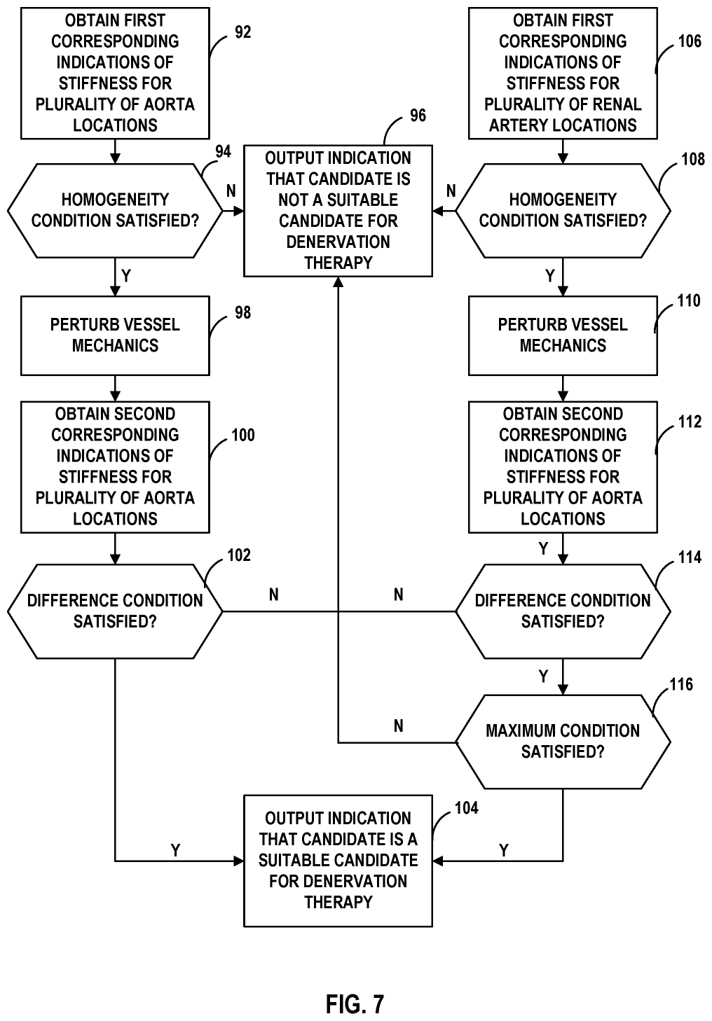

In some examples, computing device 12 may be configured to analyze vessel stiffness data associated with two or more vessels to determine whether a patient is a candidate for denervation therapy. For instance, computing device 12 may be configured to analyze vessel stiffness data associated with an aorta and a renal vessel to determine whether a patient is a candidate for renal denervation therapy. FIG. 7 is a flow diagram illustrating another example technique for operating system 10. As indicated by FIG. 7, computing device 12 may obtain a first corresponding plurality of indications of stiffness for a plurality of locations of a first vessel, which may be an aorta (92). Computing device 12 may determine whether the first corresponding plurality of indications of stiffness for the plurality of locations of the aorta locations satisfies a homogeneity condition (94). If computing device 12 determines that the homogeneity condition is not satisfied (N of 94), computing device 12 may output an indication that patient 18 is not a suitable candidate for denervation therapy (96). If computing device 12 determines that the homogeneity condition is satisfied (Y of 94), the first vessel mechanics may be perturbed (98). In some examples, the perturbation may be induced or otherwise caused by a bolus injection of a catecholamine, an acute occlusion of an inferior vena cava, a cold pressor stimulation, a Valsalva maneuver, a Müller's maneuver, a hand grip exercise, and/or the like.

Responsive to perturbation of vessel mechanics of the aorta, computing device 12 may obtain a second corresponding plurality of indications of stiffness for the plurality of locations of the aorta (100). Computing device 12 may determine whether a difference between the first corresponding plurality of indications of stiffness for the plurality of locations of the aorta and the second corresponding plurality of indications of stiffness for the plurality of locations of the aorta satisfies a difference condition (102). For example, if the difference between the first corresponding plurality of indications of stiffness for the plurality of locations of the aorta and the second corresponding plurality of indications of stiffness for the plurality of locations of the aorta is greater than or equal to the difference condition, then computing device 12 may determine that the difference condition is satisfied. In any case, if computing device 12 determines that the difference condition is not satisfied (N of 102), computing device 12 may output an indication that patient 18 is not a suitable candidate for denervation therapy (96). If computing device 12 determines that the difference condition is satisfied (Y of 102), computing device 12 may output an indication that patient 18 is a suitable candidate for denervation therapy (104).

Additionally or alternatively, computing device 12 may obtain a first corresponding plurality of indications of stiffness for a plurality of locations of a second vessel, which may be a renal artery (106). Computing device 12 may determine whether the first corresponding plurality of indications of stiffness for the plurality of locations of the renal artery satisfies a homogeneity condition (108). If computing device 12 determines that the homogeneity condition is not satisfied (N of 108), computing device 12 may output an indication that patient 18 is not a suitable candidate for denervation therapy (96). If computing device 12 determines that the homogeneity condition is satisfied (Y of 108), the second vessel mechanics may be perturbed (110). In some examples, the perturbation may be induced or otherwise caused by a bolus injection of a catecholamine, an acute occlusion of an inferior vena cava, a cold pressor stimulation, a Valsalva maneuver, a Müller's maneuver, a hand grip exercise, and/or the like.

Responsive to perturbation of vessel mechanics of the renal artery, computing device 12 may obtain a second corresponding plurality of indications of stiffness for the plurality of locations of the renal artery (112). Computing device 12 may determine whether a difference between the first corresponding plurality of indications of stiffness for the plurality of locations of the renal artery and the second corresponding plurality of indications of stiffness for the plurality of locations of the renal artery satisfies a difference condition (114). If computing device 12 determines that the difference condition is not satisfied (N of 114), computing device 12 may output an indication that patient 18 is not a suitable candidate for denervation therapy (96).

If computing device 12 determines that the difference condition is satisfied (Y of 114), computing device 12 may determine whether the second corresponding plurality of indications of stiffness for the plurality of locations of the renal artery satisfies a maximum condition (116). In some examples, computing device 12 may determine that the second plurality of corresponding indications of stiffness satisfies the maximum condition if the second plurality of corresponding indications of stiffness (or a mathematical derivation thereof, such as a mean, median, mode, etc.) is equal to or greater than a threshold value associated with the maximum condition. In any case, if computing device 12 determines that the maximum condition is not satisfied (N of 116), computing device 12 may output an indication that patient 18 is not a suitable candidate for denervation therapy (86). If computing device 12 determines that the maximum condition is satisfied (Y of 116), computing device 12 may output an indication that patient 18 is not a suitable candidate for denervation therapy (96).

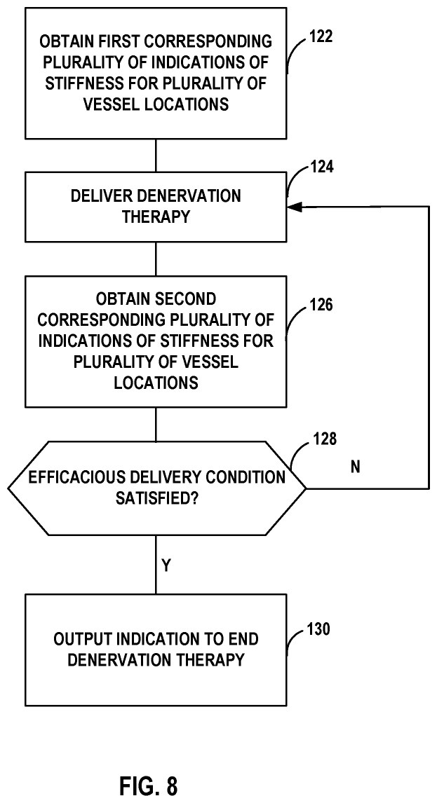

As described above, in some examples, pulse wave imaging or another measure of local vessel stiffness may be used to determine whether a denervation therapy is successful. FIG. 8 is a flow diagram illustrating another example technique for operating system 10, in which local vessel stiffness may be used to determine whether a denervation therapy is successful. As indicated by FIG. 8, computing device 12 may obtain (e.g., via data collection device 16), at a first time, a first corresponding plurality of indications of stiffness for a plurality of locations within vessel 62 (122). The first time may be prior to initiation of denervation therapy by controller 22. Controller 22 may initiate delivery of denervation therapy via denervation device 20 (124). Delivery of denervation therapy may cause a change in vessel stiffness. Computing device 12 may obtain, at a second time, a second corresponding plurality of indications of vessel stiffness (126). The second time may be subsequent to controller 22 initiating the delivery of denervation therapy.

Computing device 12 may determine whether the corresponding indications of stiffness satisfy one or more conditions (126). The one or more conditions may include a efficacy condition. If the delivery condition is satisfied (Y of 128), as evidenced by a difference between the first plurality of indications of stiffness and the second plurality of indications of stiffness being greater than a threshold difference value, computing device 12 may output an indication to end delivery of denervation therapy (128). However, if the delivery condition is not satisfied (N of 806), as evidenced by a difference between the first plurality of indications of stiffness and the second plurality of indications of stiffness being less than a threshold difference value, computing device 12 may cause controller 22 may continue delivering denervation therapy (124). Computing device 12 may iterate steps 124, 126, and 128 until the delivery condition is satisfied.

Computing device 12 may determine that the delivery condition is satisfied when the difference between the first corresponding indication of stiffness and a subsequent corresponding indication of stiffness is greater than or equal to a threshold value associated with the delivery condition. The difference may be between individual measurements within each plurality of indications (e.g., measurements associated with the same location within the vessel in each plurality of indications), between similar statistical measures of the first and second plurality of indications of vessel stiffness (e.g., mean or median), or the like. Thus, in such examples, computing device 12 may output an indication to end the delivery of denervation therapy in response to the difference between the first corresponding indications of stiffness and the second corresponding indications of stiffness being greater than or equal to the threshold value. However, if the difference between the first corresponding indications of stiffness and the second corresponding indications of stiffness is less than the threshold value, computing device 12 may cause controller 22 to continue delivering denervation therapy (124) until the difference between the first corresponding indication of stiffness and any subsequent corresponding indication of stiffness is greater than or equal to the threshold value.

In some examples, computing device 12 may be configured to obtain, for the location of the vessel and at a third time, a third corresponding indication of stiffness. The third time may be subsequent to controller 22 ending delivery of denervation therapy.

Various aspects of the techniques may enable the following examples.

Example 1: A computing device configured to: obtain, for each location of a plurality of locations of a vessel of a patient, a corresponding indication of stiffness associated with the location of the vessel; determine whether the corresponding indications of stiffness satisfy a homogeneity condition; and responsive to the computing device determining that the corresponding indications of stiffness satisfy the homogeneity condition, output an indication that the patient is a candidate for a denervation therapy.

Example 2: The computing device of example 1, wherein the plurality of corresponding indications of stiffness is a first plurality of corresponding indications of stiffness, and wherein the computing device is further configured to: obtain, for the plurality of locations of the vessel, a second plurality of corresponding indications of stiffness in response to a perturbation of blood flow through the vessel; and determine a difference between the first corresponding plurality of stiffness and the second corresponding plurality of stiffness, wherein the computing device is configured to output the indication that the patient is a candidate for the denervation therapy in response to the computing device determining that the corresponding indications of stiffness satisfy the homogeneity condition and determining that the difference satisfies a difference condition.

Example 3: The computing device of example 2, wherein the perturbation of blood flow through the vessel includes at least one of a bolus injection of a catecholamine, an acute occlusion of an inferior vena cava, or a cold pressor stimulation.

Example 4: The computing device of example 2 or 3, wherein the perturbation of blood flow through the vessel includes at least one of a Valsalva maneuver, a Müller's maneuver, or a hand grip exercise.

Example 5: The computing device of any of examples 2 through 4, wherein the computing device is configured to output the indication that the patient is a candidate for the denervation therapy in response to the computing device determining that the corresponding indications of stiffness satisfy the homogeneity condition, determining that the difference satisfies the difference condition, and determining that the second plurality of corresponding indications of stiffness satisfies a maximum condition.

Example 6: The computing device of any of examples 1 through 5, wherein the vessel is an artery.

Example 7: The computing device of example 6, wherein the artery is an aorta, a renal artery, a splanchnic artery, a hepatic artery, or a mesenteric artery.

Example 8: The computing device of any of examples 1 through 7, wherein the computing device is configured to obtain, for each location of the plurality of locations, a corresponding localized pulse wave velocity measured using pulse wave imaging.

Example 9: The computing device of any of examples 1 through 8, wherein the computing device is configured to obtain, for each location of the plurality of locations, a corresponding shear wave speed measured using ultrasound shear wave elastography.

Example 10: The computing device of any of examples 1 through 9, wherein the computing device is further configured to receive data representative of an image of the vessel.

Example 11: The computing device of example 10, wherein the image is obtained using a contrast enhanced ultrasound.

Example 12: The computing device of example 10 or 11, wherein the image provides an indication of whether at least one of plaque, extravascular calcification, aneurysm, or vessel dissection within the vessel is present.

Example 13: The computing device of any of examples 1 through 12, wherein the vessel includes a renal vessel, and wherein the denervation therapy includes renal denervation therapy.

Example 14: A system includes: a generator configured to deliver denervation therapy to a patient; a denervation device coupled to the generator; a data collection device configured to collect data indicative of vessel stiffness; and a computing device communicatively coupled to the data collection device, wherein the computing device is configured to: obtain, for a location of a vessel of the patient and at a first time, a first corresponding indication of stiffness; obtain, for the location and at a second time, a second corresponding indication of stiffness, wherein the second time is subsequent to the first time; determine a difference between the first corresponding indication of stiffness and the second corresponding indication of stiffness; and output an indication to end the delivery of denervation therapy in response to the difference satisfying a delivery condition.

Example 15: The system of example 14, wherein the first time is prior to the generator initiating delivery of denervation therapy, and wherein the second time is subsequent to the generator initiating the delivery of denervation therapy.

Example 16: The system of example 14 or 15, wherein the computing device is further configured to obtain, for the location of the vessel and at a third time, a third corresponding indication of stiffness, wherein the third time is subsequent to the generator ending delivery of denervation therapy.

Example 17: The system of any of examples 14 through 16, wherein the vessel is an artery.

Example 18: The system of any of examples 14 through 17, wherein the artery is an aorta, a renal artery, a splanchnic artery, a hepatic artery, or a mesenteric artery.

Example 19: The system of any of examples 14 through 18, wherein the data collection device is configured to measure, for the location, a localized pulse wave velocity, and wherein the computing device is configured to obtain, from the data collection device and for the location, a first corresponding localized pulse wave velocity and a second corresponding localized pulse wave velocity.

Example 20: The system of example 19, wherein the computing device is configured to obtain the first corresponding localized pulse wave velocity and the second corresponding localized pulse wave velocity by using pulse wave imaging.

Example 21: The system of any of examples 14 through 20, wherein the data collection device is configured to measure, for the location, a shear wave speed, and wherein the computing device is configured to obtain, from the data collection device, a first corresponding shear wave speed and a second corresponding shear wave speed.

Example 22: The system of any of examples 14 through 21, wherein the computing device is configured to obtain the first corresponding shear wave speed and a second corresponding shear wave speed by using ultrasound shear wave elastography.

Example 23: The system of any of examples 14 through 22, wherein the computing device is further configured to: receive data representative of an image of the vessel; and discriminate, based on the image of the vessel, between reversible vessel stiffness and irreversible vessel stiffness.

Example 24: The system of any of examples 14 through 23, wherein the image is obtained using a contrast enhanced ultrasound.

Example 25: The method of example 24, wherein discriminating between reversible vessel stiffness and irreversible vessel stiffness is based on identifying at least one of plaque, extravascular calcification, aneurysm, or vessel dissection within the vessel based on the image of the vessel.

Example 26: The computing device of any of examples 14 through 25, wherein the vessel includes a renal vessel, and wherein the denervation therapy includes renal denervation therapy.

Example 27: A method including: obtaining, by a computing device, for each location of a plurality of locations of a vessel of a patient, a corresponding indication of a stiffness of the associated with the location of the vessel; determining, by the computing device, whether the corresponding indications of stiffness satisfy a homogeneity condition; and in response to determining that the corresponding indications of stiffness satisfy the homogeneity condition, outputting, by the computing device, an indication that the patient is a candidate for a denervation therapy.

Example 28: The method of example 27, wherein the plurality of corresponding indications of stiffness is a first plurality of corresponding indications of stiffness, and wherein the method further includes: obtaining, by the computing device, for the plurality of locations of the vessel, a second plurality corresponding indications of stiffness in response to a perturbation of blood flow through the vessel; and determining, by the computing device, a difference between the first corresponding plurality of stiffness and the second corresponding plurality of stiffness; and wherein outputting the indication that the patient is a candidate for the denervation therapy includes outputting the indication that the patient is a candidate for the denervation therapy in response to determining that the corresponding indications of stiffness satisfy the homogeneity condition and determining that the difference satisfies a difference condition.

Example 29: The method of example 28, wherein the perturbation of blood flow through the vessel includes at least one of a bolus injection of a catecholamine, an acute occlusion of an inferior vena cava, or a cold pressor stimulation.

Example 30: The method of example 28 or 29, wherein the perturbation of blood flow through the vessel includes at least one of a Valsalva maneuver, a Müller's maneuver, or a hand grip exercise.

Example 31: The method of any of examples 28 through 30, wherein outputting the indication that the patient is a candidate for the denervation therapy includes outputting the indication that the patient is a candidate for the denervation therapy in response to determining that the corresponding indications of stiffness satisfy the homogeneity condition, determining that the difference satisfies the difference condition, and determining that the second plurality of corresponding indications of stiffness satisfies a maximum condition.

Example 32: The method of any of examples 27 through 31, wherein the vessel is an artery.

Example 33: The method of example 32, wherein the artery is an aorta, a renal artery, a splanchnic artery, a hepatic artery, or a mesenteric artery.

Example 34. The method of any of examples 27 through 33, wherein obtaining, by the computing device, the corresponding indication of a stiffness associated with the location includes receiving, by the computing device, for each location of the plurality of locations, a corresponding localized pulse wave velocity measured using pulse wave imaging.

Example 35: The method of any of examples 27 through 34, wherein obtaining, by the computing device, the corresponding indication of a stiffness associated with the location includes receiving, for each location of the plurality of locations, a corresponding shear wave speed measured using ultrasound shear wave elastography.

Example 36: The method of any of examples 27 through 35, further including: receiving, by the computing device, data representative of an image of the vessel; and discriminating, by the computing device, based on the image of the vessel, between reversible vessel stiffness and irreversible vessel stiffness.

Example 37: The method of example 36, wherein the image is obtained using a contrast enhanced ultrasound.

Example 38: The method of example 36 or 37, wherein discriminating between reversible vessel stiffness and irreversible vessel stiffness is based on identifying at least one of plaque, extravascular calcification, aneurysm, or vessel dissection within the vessel based on the image of the vessel.

Example 39: The method of any of examples 27 through 38, wherein the vessel includes a renal vessel, and wherein the denervation therapy includes renal denervation therapy.

Example 40: A method including: determining, for a location of a vessel of a patient and at a first time, a first corresponding indication of stiffness; delivering denervation therapy to the patient; determining, for the location of the vessel and at a second time, a second corresponding indication of stiffness, wherein the second time is subsequent to the first time; determining a difference between the first corresponding indication of stiffness and the second corresponding indication of stiffness; and ending the delivery of renal denervation therapy in response to the difference satisfying a threshold value.

Example 41: The method of example 40, wherein the first time is prior to an initiation of the delivery of renal denervation therapy, and wherein the second time is subsequent to the initiation of the delivery of renal denervation therapy.

Example 42: The method of example 40 or 41, further including determining, for the location of the vessel and at a third time, a third corresponding stiffness, wherein the third time is subsequent to ending delivery of denervation therapy.

Example 43: The method of any of examples 40 through 42, wherein the vessel is an artery.

Example 44: The method of example 43, wherein the artery is an aorta, a renal artery, a splanchnic artery, a hepatic artery, or a mesenteric artery.

Example 45: The method of any of examples 40 through 44, wherein determining the first corresponding indication of stiffness and the second corresponding indication of stiffness includes measuring, for the location, a first corresponding localized pulse wave velocity and a second corresponding localized pulse wave velocity.

Example 46: The method of example 45, wherein measuring the first corresponding localized pulse wave velocity and the second corresponding localized pulse wave velocity includes using pulse wave imaging.

Example 47: The method of any of examples 40 through 46, wherein determining the first corresponding indication of stiffness and the second corresponding indication of stiffness includes measuring, for the location, a first corresponding shear wave speed and a second corresponding shear wave speed.

Example 48: The method of example 47, wherein measuring the first corresponding shear wave speed and the second corresponding shear wave speed includes using ultrasound shear wave elastography.

Example 49: The method of any of examples 40 through 48, further including: receiving, by the computing device, data representative of an image of the vessel; and discriminating, by the computing device and based on the image of the vessel, between reversible vessel stiffness and irreversible vessel stiffness.

Example 50: The method of example 49, wherein the image is obtained using a contrast enhanced ultrasound.

Example 51: The method of example 50, wherein discriminating between reversible vessel stiffness and irreversible vessel stiffness is based on visualization of at least one of plaque, extravascular calcification, aneurysm, or vessel dissection within the vessel based on the image of the vessel.

Example 52: The method of any of examples 40 through 51, wherein the vessel includes a renal vessel, and wherein the denervation therapy includes renal denervation therapy.

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also perform one or more of the techniques of this disclosure.

Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described. These and other examples are within the scope of the following claims.

Further disclosed herein is the subject-matter of the following clauses:
1. A computing device configured to:
   obtain, for each location of a plurality of locations of a vessel of a patient, a corresponding indication of stiffness associated with the location of the vessel;
   determine whether the corresponding indications of stiffness satisfy a homogeneity condition; and
   responsive to the computing device determining that the corresponding indications of stiffness satisfy the homogeneity condition, output an indication that the patient is a candidate for a denervation therapy.
2. The computing device of clause 1, wherein the plurality of corresponding indications of stiffness is a first plurality of corresponding indications of stiffness, and wherein the computing device is further configured to:
   obtain, for the plurality of locations of the vessel, a second plurality of corresponding indications of stiffness in response to a perturbation of blood flow through the vessel; and
   determine a difference between the first corresponding plurality of stiffness and the second corresponding plurality of stiffness,
   wherein the computing device is configured to output the indication that the patient is a candidate for the denervation therapy in response to the computing device determining that the corresponding indications of stiffness satisfy the homogeneity condition and determining that the difference satisfies a difference condition.
3. The computing device of clause 2, wherein the perturbation of blood flow through the vessel comprises at least one of a bolus injection of a catecholamine, an acute occlusion of an inferior vena cava, or a cold pressor stimulation.
4. The computing device of clause 2 or 3, wherein the perturbation of blood flow through the vessel comprises at least one of a Valsalva maneuver, a Müller's maneuver, or a hand grip exercise.
5. The computing device of any of clauses 2 through 4, wherein the computing device is configured to output the indication that the patient is a candidate for the denervation therapy in response to the computing device determining that the corresponding indications of stiffness satisfy the homogeneity condition, determining that the difference satisfies the difference condition, and determining that the second plurality of corresponding indications of stiffness satisfies a maximum condition.
6. The computing device of any of clauses 1 through 5, wherein the vessel is an artery.
7. The computing device of clause 6, wherein the artery is an aorta, a renal artery, a splanchnic artery, a hepatic artery, or a mesenteric artery.
8. The computing device of any of clauses 1 through 7, wherein the computing device is configured to obtain, for each location of the plurality of locations, a corresponding localized pulse wave velocity measured using pulse wave imaging.
9. The computing device of any of clauses 1 through 8, wherein the computing device is configured to obtain, for each location of the plurality of locations, a corresponding shear wave speed measured using ultrasound shear wave elastography.
10. The computing device of any of clauses 1 through 9, wherein the computing device is further configured to receive data representative of an image of the vessel.
11. The computing device of clause 10, wherein the image is obtained using a contrast enhanced ultrasound.
12. The computing device of clause 10 or 11, wherein the image provides an indication of whether at least one of plaque, extravascular calcification, aneurysm, or vessel dissection within the vessel is present.
13. The computing device of any of clauses 1 through 12, wherein the vessel comprises a renal vessel, and wherein the denervation therapy comprises renal denervation therapy.
14. A system comprising:
   a generator configured to deliver denervation therapy to a patient;
   a denervation device coupled to the generator;
   a data collection device configured to collect data indicative of vessel stiffness; and
   a computing device communicatively coupled to the data collection device, wherein the computing device is configured to:
      obtain, for a location of a vessel of the patient and at a first time, a first corresponding indication of stiffness;
      obtain, for the location and at a second time, a second corresponding indication of stiffness, wherein the second time is subsequent to the first time;
      determine a difference between the first corresponding indication of stiffness and the second corresponding indication of stiffness; and
      output an indication to end the delivery of denervation therapy in response to the difference satisfying a delivery condition.
15. The system of clause 14, wherein the first time is prior to the generator initiating delivery of denervation therapy, and wherein the second time is subsequent to the generator initiating the delivery of denervation therapy.
16. The system of clause 14 or 15, wherein the computing device is further configured to obtain, for the location of the vessel and at a third time, a third corresponding indication of stiffness, wherein the third time is subsequent to the generator ending delivery of denervation therapy.
17. The system of any of clauses 14 through 16, wherein the vessel is an artery.
18. The system of any of clauses 14 through 17, wherein the artery is an aorta, a renal artery, a splanchnic artery, a hepatic artery, or a mesenteric artery.
19. The system of any of clauses 14 through 18, wherein the data collection device is configured to measure, for the location, a localized pulse wave velocity, and wherein the computing device is configured to obtain, from the data collection device and for the location, a first corresponding localized pulse wave velocity and a second corresponding localized pulse wave velocity.
20. The system of clause 19, wherein the computing device is configured to obtain the first corresponding localized pulse wave velocity and the second corresponding localized pulse wave velocity by using pulse wave imaging.
21. The system of any of clauses 14 through 20, wherein the data collection device is configured to measure, for the location, a shear wave speed, and wherein the computing device is configured to obtain, from the data collection device, a first corresponding shear wave speed and a second corresponding shear wave speed.
22. The system of any of clauses 14 through 21, wherein the computing device is configured to obtain the first corresponding shear wave speed and a second corresponding shear wave speed by using ultrasound shear wave elastography.
23. The system of any of clauses 14 through 22, wherein the computing device is further configured to:
   receive data representative of an image of the vessel; and
   discriminate, based on the image of the vessel, between reversible vessel stiffness and irreversible vessel stiffness.
24. The system of any of clauses 14 through 23, wherein the image is obtained using a contrast enhanced ultrasound.
25. The method of clause 24, wherein discriminating between reversible vessel stiffness and irreversible vessel stiffness is based on identifying at least one of plaque, extravascular calcification, aneurysm, or vessel dissection within the vessel based on the image of the vessel.
26. The computing device of any of clauses 14 through 25, wherein the vessel comprises a renal vessel, and wherein the denervation therapy comprises renal denervation therapy.
27. A method comprising:
   obtaining, by a computing device, for each location of a plurality of locations of a vessel of a patient, a corresponding indication of a stiffness of the associated with the location of the vessel;
   determining, by the computing device, whether the corresponding indications of stiffness satisfy a homogeneity condition; and
   in response to determining that the corresponding indications of stiffness satisfy the homogeneity condition, outputting, by the computing device, an indication that the patient is a candidate for a denervation therapy.
28. The method of clause 27, wherein the plurality of corresponding indications of stiffness is a first plurality of corresponding indications of stiffness, and wherein the method further comprises:
   obtaining, by the computing device, for the plurality of locations of the vessel, a second plurality corresponding indications of stiffness in response to a perturbation of blood flow through the vessel; and
   determining, by the computing device, a difference between the first corresponding plurality of stiffness and the second corresponding plurality of stiffness; and
   wherein outputting the indication that the patient is a candidate for the denervation therapy comprises outputting the indication that the patient is a candidate for the denervation therapy in response to determining that the corresponding indications of stiffness satisfy the homogeneity condition and determining that the difference satisfies a difference condition.
29. The method of clause 28, wherein the perturbation of blood flow through the vessel comprises at least one of a bolus injection of a catecholamine, an acute occlusion of an inferior vena cava, or a cold pressor stimulation.
30. The method of clause 28 or 29, wherein the perturbation of blood flow through the vessel comprises at least one of a Valsalva maneuver, a Müller's maneuver, or a hand grip exercise.
31. The method of any of clauses 28 through 30, wherein outputting the indication that the patient is a candidate for the denervation therapy comprises outputting the indication that the patient is a candidate for the denervation therapy in response to determining that the corresponding indications of stiffness satisfy the homogeneity condition, determining that the difference satisfies the difference condition, and determining that the second plurality of corresponding indications of stiffness satisfies a maximum condition.
32. The method of any of clauses 27 through 31, wherein the vessel is an artery.
33. The method of clause 32, wherein the artery is an aorta, a renal artery, a splanchnic artery, a hepatic artery, or a mesenteric artery.
34. The method of any of clauses 27 through 33, wherein obtaining, by the computing device, the corresponding indication of a stiffness associated with the location comprises receiving, by the computing device, for each location of the plurality of locations, a corresponding localized pulse wave velocity measured using pulse wave imaging.
35. The method of any of clauses 27 through 34, wherein obtaining, by the computing device, the corresponding indication of a stiffness associated with the location comprises receiving, for each location of the plurality of locations, a corresponding shear wave speed measured using ultrasound shear wave elastography.
36. The method of any of clauses 27 through 35, further comprising:
   receiving, by the computing device, data representative of an image of the vessel; and
   discriminating, by the computing device, based on the image of the vessel, between reversible vessel stiffness and irreversible vessel stiffness.
37. The method of clause 36, wherein the image is obtained using a contrast enhanced ultrasound.
38. The method of clause 36 or 37, wherein discriminating between reversible vessel stiffness and irreversible vessel stiffness is based on identifying at least one of plaque, extravascular calcification, aneurysm, or vessel dissection within the vessel based on the image of the vessel.
39. The method of any of clauses 27 through 38, wherein the vessel comprises a renal vessel, and wherein the denervation therapy comprises renal denervation therapy.
40. A method comprising:
   determining, for a location of a vessel of a patient and at a first time, a first corresponding indication of stiffness;
   delivering denervation therapy to the patient;
   determining, for the location of the vessel and at a second time, a second corresponding indication of stiffness, wherein the second time is subsequent to the first time;
   determining a difference between the first corresponding indication of stiffness and the second corresponding indication of stiffness; and
   ending the delivery of renal denervation therapy in response to the difference satisfying a threshold value.
41. The method of clause 40, wherein the first time is prior to an initiation of the delivery of renal denervation therapy, and wherein the second time is subsequent to the initiation of the delivery of renal denervation therapy.
42. The method of clause 40 or 41, further comprising determining, for the location of the vessel and at a third time, a third corresponding stiffness, wherein the third time is subsequent to ending delivery of denervation therapy.
43. The method of any of clauses 40 through 42, wherein the vessel is an artery.
44. The method of clause 43, wherein the artery is an aorta, a renal artery, a splanchnic artery, a hepatic artery, or a mesenteric artery.
45. The method of any of clauses 40 through 44, wherein determining the first corresponding indication of stiffness and the second corresponding indication of stiffness comprises measuring, for the location, a first corresponding localized pulse wave velocity and a second corresponding localized pulse wave velocity.
46. The method of clause 45, wherein measuring the first corresponding localized pulse wave velocity and the second corresponding localized pulse wave velocity comprises using pulse wave imaging.
47. The method of any of clauses 40 through 46, wherein determining the first corresponding indication of stiffness and the second corresponding indication of stiffness comprises measuring, for the location, a first corresponding shear wave speed and a second corresponding shear wave speed.
48. The method of clause 47, wherein measuring the first corresponding shear wave speed and the second corresponding shear wave speed comprises using ultrasound shear wave elastography.
49. The method of any of clauses 40 through 48, further comprising:
   receiving, by the computing device, data representative of an image of the vessel; and
   discriminating, by the computing device and based on the image of the vessel, between reversible vessel stiffness and irreversible vessel stiffness.
50. The method of clause 49, wherein the image is obtained using a contrast enhanced ultrasound.
51. The method of clause 50, wherein discriminating between reversible vessel stiffness and irreversible vessel stiffness is based on visualization of at least one of plaque, extravascular calcification, aneurysm, or vessel dissection within the vessel based on the image of the vessel.
52. The method of any of clauses 40 through 51, wherein the vessel comprises a renal vessel, and wherein the denervation therapy comprises renal denervation therapy.

1. A computing device configured to:
obtain, for each location of a plurality of locations of a vessel of a patient, a corresponding indication of stiffness associated with the location of the vessel;
determine whether the corresponding indications of stiffness satisfy a homogeneity condition; and
responsive to the computing device determining that the corresponding indications of stiffness satisfy the homogeneity condition, output an indication that the patient is a candidate for a denervation therapy.

2. The computing device of claim 1, wherein the plurality of corresponding indications of stiffness is a first plurality of corresponding indications of stiffness, and wherein the computing device is further configured to:
obtain, for the plurality of locations of the vessel, a second plurality of corresponding indications of stiffness in response to a perturbation of blood flow through the vessel; and
determine a difference between the first plurality of corresponding indications of stiffness and the second plurality of corresponding indications of stiffness,
wherein the computing device is configured to output the indication that the patient is the candidate for the denervation therapy in response to the computing device:
determining that the first plurality of corresponding indications of stiffness satisfy the homogeneity condition; and
determining that the difference satisfies a difference condition.

3. The computing device of claim 2, wherein the perturbation of blood flow through the vessel comprises at least one of:
at least one of a bolus injection of a catecholamine, an acute occlusion of an inferior vena cava, or a cold pressor stimulation; or
at least one of a Valsalva maneuver, a Müller's maneuver, or a hand grip exercise.

4. The computing device of claim 1, wherein the vessel is an artery, wherein the artery is an aorta, a renal artery, a splanchnic artery, a hepatic artery, or a mesenteric artery.

5. The computing device of claim 1, wherein the computing device is configured to obtain, for each location of the plurality of locations, at least one of:
a corresponding localized pulse wave velocity measured using pulse wave imaging; or
a corresponding shear wave speed measured using ultrasound shear wave elastography.

6. The computing device of claim 1, wherein the computing device is further configured to receive data representative of an image of the vessel, wherein the image is obtained using a contrast enhanced ultrasound, and wherein the image provides an indication of whether at least one of plaque, extravascular calcification, aneurysm, or vessel dissection within the vessel is present.

7. The computing device of claim 1, wherein the vessel comprises a renal vessel, and wherein the denervation therapy comprises renal denervation therapy.

8. A system comprising:
a generator configured to deliver denervation therapy to a patient;
a denervation device coupled to the generator;
a data collection device configured to collect data indicative of vessel stiffness; and
a computing device communicatively coupled to the data collection device, wherein the computing device is configured to:
obtain, for a location of a vessel of the patient and at a first time, a first corresponding indication of stiffness;
obtain, for the location and at a second time, a second corresponding indication of stiffness, wherein the second time is subsequent to the first time;
determine a difference between the first corresponding indication of stiffness and the second corresponding indication of stiffness; and
output an indication to end the delivery of denervation therapy in response to the difference satisfying a delivery condition.

9. The system of claim 8, wherein the first time is prior to the generator initiating the delivery of denervation therapy, and wherein the second time is subsequent to the generator initiating the delivery of denervation therapy.

10. The system of claim 8, wherein the data collection device is configured to measure, for the location, a localized pulse wave velocity, and wherein the computing device is configured to obtain, from the data collection device and for the location, a first corresponding localized pulse wave velocity and a second corresponding localized pulse wave velocity.

11. The system of claim 8, wherein the data collection device is configured to measure, for the location, a shear wave speed, and wherein the computing device is configured to obtain, from the data collection device, a first corresponding shear wave speed and a second corresponding shear wave speed.

12. The system of claim 8, wherein the image is obtained using a contrast enhanced ultrasound, wherein discriminating between reversible vessel stiffness and irreversible vessel stiffness is based on identifying at least one of plaque, extravascular calcification, aneurysm, or vessel dissection within the vessel based on the image of the vessel.

13. A method comprising:
obtaining, by a computing device, for each location of a plurality of locations of a vessel of a patient, a corresponding indication of a stiffness of the associated with the location of the vessel;
determining, by the computing device, whether the corresponding indications of stiffness satisfy a homogeneity condition; and
in response to determining that the corresponding indications of stiffness satisfy the homogeneity condition, outputting, by the computing device, an indication that the patient is a candidate for a denervation therapy.

14. The method of claim 13, wherein the plurality of corresponding indications of stiffness is a first plurality of corresponding indications of stiffness, and wherein the method further comprises:
obtaining, by the computing device, for the plurality of locations of the vessel, a second plurality corresponding indications of stiffness in response to a perturbation of blood flow through the vessel; and
determining, by the computing device, a difference between the first plurality of corresponding indications of stiffness and the second plurality of corresponding indications of stiffness; and
wherein outputting the indication that the patient is a candidate for the denervation therapy comprises outputting the indication that the patient is a candidate for the denervation therapy in response to determining that the first plurality of corresponding indications of stiffness satisfy the homogeneity condition and determining that the difference satisfies a difference condition.

15. The method of claim 14, wherein the perturbation of blood flow through the vessel comprises at least one of:
at least one of a bolus injection of a catecholamine, an acute occlusion of an inferior vena cava, or a cold pressor stimulation; or
at least one of a Valsalva maneuver, a Müller's maneuver, or a hand grip exercise.

16. The method of claim 14, wherein outputting the indication that the patient is a candidate for the denervation therapy comprises outputting the indication that the patient is the candidate for the denervation therapy in response to determining that the corresponding indications of stiffness satisfy the homogeneity condition, determining that the difference satisfies the difference condition, and determining that the second plurality of corresponding indications of stiffness satisfies a maximum condition.

17. The method of claim 13, wherein the vessel is an artery, wherein the artery is an aorta, a renal artery, a splanchnic artery, a hepatic artery, or a mesenteric artery.

18. The method of claim 13, wherein obtaining, by the computing device, the corresponding indication of the stiffness associated with the location comprises receiving, by the computing device, for each location of the plurality of locations, at least one of:
a corresponding localized pulse wave velocity measured using pulse wave imaging; or
a corresponding shear wave speed measured using ultrasound shear wave elastography.

19. The method of claim 13, further comprising:
receiving, by the computing device, data representative of an image of the vessel; and
discriminating, by the computing device, based on the image of the vessel, between reversible vessel stiffness and irreversible vessel stiffness, wherein discriminating between reversible vessel stiffness and irreversible vessel stiffness is based on identifying at least one of plaque, extravascular calcification, aneurysm, or vessel dissection within the vessel based on the image of the vessel.

20. The computing device of claim 2, wherein the computing device is configured to output the indication that the patient is the candidate for the denervation therapy in response to the computing device determining that the first and second pluralities of corresponding indications of stiffness satisfy the homogeneity condition, determining that the difference between the first and second pluralities of corresponding indications of stiffness satisfies the difference condition, and determining that the second plurality of corresponding indications of stiffness satisfies a maximum condition.
